(19) United States
(12) Patent Application Publication
(10) Pub. No.: US 2006/0224070 A1
(43) Pub. Date: Oct. 5, 2006

(54) System and method for non-invasive cardiovascular assessment from supra-systolic signals obtained with a wideband external pulse transducer in a blood pressure cuff

(76) Inventors: Nigel E. Sharrock, New York, NY (US); Andrew Lowe, Meadowbank (NZ); Daniel Norberto Roldan, Remuera (NZ)

Correspondence Address: MILDE & HOFFBERG, LLP, 10 BANK STREET, SUITE 460, WHITE PLAINS, NY, 10606, US

(21) Appl. No.: 11/358,283

(22) Filed: Feb. 21, 2006

Related U.S. Application Data
(60) Provisional application No. 60/668,336, filed on Apr. 5, 2005.
(60) Provisional application No. 60/673,973, filed on Apr. 22, 2005.
(60) Provisional application No. 60/673,974, filed on Apr. 22, 2005.
(60) Provisional application No. 60/673,975, filed on Apr. 22, 2005.

(51) Int. Cl. A61B 5/02

(52) U.S. Cl. 600/500

(57) ABSTRACT

A method and apparatus are disclosed for non-invasively determining a cardiovascular status of a patient. Cardiac pulse waveforms associated with the peripheral artery are monitored during a plurality of cardiac ejection cycles, using a wideband external pulse transducer. The waveforms are analyzed to obtain information relating to the patient's Augmentation index (AI), cardiac performance, and/or cardiac stroke volume.

### CROSS-REFERENCE TO RELATED APPLICATIONS

This application claims priority from the U.S. provisional patent application No. 60/668,336 filed Apr. 5, 2005, and the three U.S. provisional patent applications Nos. 60/673,973; 60/673,974 and 60/673,975, all filed on Apr. 22, 2005. This application also contains subject matter related to that disclosed in the U.S. patent application Ser. No. 10/221,530 filed Sep. 13, 2002 and entitled “Non-Invasive Measurement of Suprasystolic Signals” (Publication No. US 2003/0040675 A1) which is incorporated herein by reference.

### FIELD OF THE INVENTION

This invention relates to non-invasive cardiovascular assessment of a patient based on the evaluation of pressure wave signals obtained by means of a low frequency, wideband electrical transducer or sensor disposed in, on or under the Korotkoff arm cuff of a sphygmomanometer.

More particularly, the invention relates to the non-invasive assessment of aortic compliance and other cardiovascular parameters by analyzing signals obtained from a sensor of this type.

### BACKGROUND OF THE INVENTION

The signals recorded with a sensor placed beneath a blood pressure cuff are termed “supra-systolic” signals if the cuff pressure is above the subject's systolic blood pressure. In addition, signals can be recorded when the cuff pressure is below systolic pressure. In all cases, the signals result from pressure energy transmissions and are dependent upon the subject's physiology.

When the heart pumps, a pressure gradient is generated within the cardiovascular system. This results in pulse pressure waves traveling peripherally from the heart through the arteries. Like any wave, they reflect back off a surface or other change in impedance. Arterial pulse waves reflect back from both the peripheral circulation and from the distal aorta when it becomes less compliant (Murgo, Westerhof et al. 1980; Latham, Westerhof et al. 1985). These reflection waves are identifiable in arterial pressure tracings, but the exact timing and magnitude of the waves are difficult to discern. Nevertheless, they have been the basis of several commercial systems to assess reflectance waves. These systems measure arterial contours using applanation tonometry from the radial artery.

If a low frequency sensor is placed over the brachial artery beneath a blood pressure cuff and the cuff is inflated above systole, supra-systolic signals can be recorded (Blank, West et al. 1988; Hirai, Sasayama et al. 1989; Denby, Mallows et al. 1994). An idealized supra-systolic signal for one heart beat is shown in FIG. 1. These signals contain frequency components of less than 20 Hertz, which are non-audible. Supra-systolic low frequency signals provide clear definition of three distinct waves: an incident wave corresponding to the pulse wave and two subsequent waves. Blank (Blank 1996) proposed that the second wave emanated from the periphery and the relative amplitude of this wave to the incident wave (K1R) was a measure of peripheral vascular resistance (PVR). He proposed a constant such that PVR could be measured from the ratio of the incident to the first reflectance wave. See, also, U.S. Pat. No. 5,913,826, which is incorporated herein by reference in its entirety.

The second supra-systolic wave is, in fact, a reflectance wave from the distal abdominal aorta—most likely originating from the bifurcation of the aorta and not from the peripheral circulation as proposed by Blank. This has been verified in human experiments (Murgo, Westerhof et al. 1980; Latham, Westerhof et al. 1985) and in studies using pulse wave velocity (PWV) measurements. The relative amplitude of the first reflectance wave is now believed to be a measure of the stiffness, compliance, or elasticity of the abdominal aorta rather than peripheral resistance.

In the clinical experiments upon which Blank relied to formulate his hypothesis, changes in compliance were induced with epinephrine and epidural anesthesia. The changes in compliance were accompanied by changes in peripheral resistance. Thus, he saw a relationship between his K1R and PVR, but it was a co-variable and not a true association.

The third wave occurs at the beginning of diastole and is believed to be a reflection wave from the peripheral circulation. As such, it is a measure of peripheral vasoconstriction with superimposed secondary reflections. Supra-systolic signals can be utilized to measure compliance by relating the amplitude of the first wave (incident or SS1) to the amplitude of the second (aortic reflection or SS2) wave. The degree of vasoconstriction can be assessed by measuring the amplitude of the diastolic or third wave (SS3 wave) and relating it to the SS1 wave. Amplitudes, areas under the curves, or other values calculated from the waves can be utilized. Data has been analyzed by measuring amplitudes, ratios of amplitudes and time delays between waves.

Augmentation Index (AI) has become recognized as an important marker of cardiovascular disease. It increases with age, hypertension and atherosclerosis. Through ventricular-vascular coupling, AI is a marker of ventricular (cardiac) hypertrophy—stiffness or diastolic dysfunction. Thus, this single measure gives an indication of the health of the whole cardiovascular system. AI is measured from an aortic pressure tracing (FIG. 8) as follows: The amplitude of the augmentation wave (Ps−Pi) is divided by the amplitude of the incident plus reflection wave (Ps−Pd). The ratio is multiplied by 100 to give a percentage.
Aortic Augmentation Index (AAI)=(Ps−Pi)/(Ps−Pd)×100 (1)

Measurements of aortic pressure can only be made in the cardiac catheterization laboratory so other non-invasive means of assessing it have been developed. Two have been described. Firstly, using tonometry on the carotid artery, a waveform can be measured which identifies the initial and late systolic peaks. A carotid augmentation index (CAI) is measured. Secondly, tonometry of the radial artery likewise provides a signal, which can be transformed to provide a measure of aortic augmentation index (AAI).

### SUMMARY OF THE INVENTION

The relationship between the aortic pressure and brachial arterial wideband supra-systolic pressure trace can be understood and a correction formula derived from a comparison between the two, both on an individual and/or on a population basis, enabling a Brachial Artery Augmentation Index (AAI) and a brachial artery derived AAI to be measured.

The present invention therefore provides a system for measuring peripheral arterial signals, e.g. of the brachial artery, using a wideband external pulse transducer disposed in, on or under a blood pressure cuff, and a processor, receiving the signals from the transducer, and processing these signals to determine distortions present in the transducer waveform with respect to an inferred original aortic waveform.

A cuff is inflated to a supra-systolic pressure, such as 15-150 mm Hg above a systolic pressure, preferably about 30 mm above the systolic pressure, measuring with a pressure transducer having sufficient bandwidth to capture detailed waveform information, for example from 0.1 to 1000 Hz, and analyzing the waveform to infer an aortic pressure waveform. Various corrections may be applied to the inference, both personal to the subject, and based on population studies, to correct for aberrations. In a preferred embodiment, a model of the patient is formulated, wherein a set of parameters, which may be generally orthogonal (e.g., parameters having low interactivity) or correlated to available clinical measurements, describe elements of the model. These parameters may then be used to populate the model, or the model used to estimate the parameters. By employing a physiological model, and analyzing the values of the parameters, as well as their responsivity to various factors, clinical conclusions are facilitated.

This inferred waveform may then be used for a number of purposes, including analyzing cardiac function, analyzing the central and/or peripheral arterial system, or for analyzing the cardiovascular system as a whole.

Another embodiment of the invention employs an algorithm for extracting features from the pressure waveform (or, for example, the model constructed from the data), which may be multivariate or complex. In any case, the parameter(s) or features may be used as diagnostic, prognostic, or therapeutic indices. Thus, if the parameter corresponds to a therapeutic target of a drug, the parameter may be monitored, and drug use titrated for its desired effect on the cardiovascular system.

Stimuli may also be used to excite various responses in the system, for example a cold pressor stimulus, which may allow more accurate or detailed analysis of the pressure data.

Thus, the present invention provides means for extracting useful parameters of central and peripheral cardiovascular system performance, without requiring a direct measurement of waveforms from the heart or aorta.

A reliable system may therefore be provided to acquire supra-systolic signals from patients, a method to analyze the signals, and clinical applications for the signals. The system consists of a low frequency transducer placed in, on or beneath a blood pressure cuff or similar device, placed around a patient's arm. The signals are conditioned and, if necessary, amplified, passed through an analog to digital converter and transferred to a computer or processor for analysis. Analyzed signals will be stored, presented on a screen numerically or graphically. Data can be stored or transmitted to databases or other health care facilities.

A variety of vibration transducers can be used. The transducer must be able to sense dynamic signals as low as about 0.1 Hertz and be sturdy enough to withstand repeated use under external pressures of about 300 mm Hg. For example, a suitable commercially available piezoelectric transducer consists of two adjacent sensors approximately 1.5 cm in diameter. The transducer is placed along the axis of the brachial artery providing proximal (closer to the heart) and distal signals. Preferably only one sensor is used. However, an alternative is to use an array of sensors to aid in noise elimination or other signal processing in certain clinical environments. Another possibility is to incorporate inexpensive sensors into a disposable blood pressure cuff to create a disposable product suitable for critical care environments where infection control is important.

According to the invention, it is possible to simplify the assessment of stroke volume and/or blood volume and/or other indicators of cardiovascular status and/or to improve the accuracy of such indicators.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a graph of idealized supra-systolic signal for one heartbeat obtained from a patient.

FIG. 2 is a diagram showing the supra-systolic pulse wave transit paths resulting in the signal of FIG. 1.

FIG. 3a is a diagram illustrating the positioning of blood pressure cuff with a wideband external pressure (WEP) transducer arranged on a patient's arm to obtain the signal of FIG. 1.

FIG. 3b is a cross-sectional view of the blood pressure cuff of FIG. 3a.

FIG. 4 is a graph showing a sample determination of area under the SS1 peak of a supra-systolic signal from a patient.

FIG. 5 is an example graph of supra-systolic signal versus time over an inspiratory/expiratory cycle of a patient breathing normally.

FIG. 6 is a graph of supra-systolic signal versus time over an inspiratory/expiratory cycle of a patient during labored breathing.

FIG. 7 is a schematic block diagram of apparatus in accordance with a preferred embodiment of the present invention.

FIG. 8 shows a pressure trace from the ascending aorta using the apparatus of the present invention.

FIG. 9 shows a supra-systolic signal with designations of its inflection points.

FIG. 10 shows overlaid traces of a pressure trace from the ascending aorta and the supra-systolic signal, using a wideband external pressure (WEP) transducer.

FIG. 11 shows a WEP transducer signal and cuff pressure on an upper axis, and an expanded WEP tracing on a lower axis, evidencing a medium Augmentation Index.

FIG. 12 is a diagram similar to FIG. 11, with an expanded WEP tracing evidencing a low Augmentation Index.

FIG. 13 is a diagram similar to FIG. 11, with an expanded WEP tracing evidencing a high Augmentation Index.

FIG. 14 is a diagram similar to FIG. 11, with an expanded WEP tracing obtained before a hand is cooled with ice.

FIG. 15 is a diagram similar to FIG. 11, with an expanded WEP tracing obtained after a hand is cooled with ice.

FIG. 16 is a diagram similar to FIG. 11, with an expanded WEP tracing with dropped heartbeats.

FIG. 17 is a diagram similar to FIG. 11, with an expanded WEP tracing evidencing varying beat-to-beat rates.

FIG. 18 is a diagram similar to FIG. 11, with an expanded WEP tracing wherein both the beat-to-beat rate and the configuration of the waves vary.

FIG. 19 is a diagram similar to FIG. 11, with an expanded WEP tracing showing large variations in the wave configuration.

FIGS. 20-23 are diagrams similar to FIG. 11, with expanded WEP tracings obtained from a succession of patients with progressively deteriorating, diastolic heart failure.

FIGS. 24-27 are diagrams similar to FIG. 11, with expended WEP tracings obtained from a young patient, a middle-aged patient and two older patients, respectively, illustrating the importance of dt1-2.

### DETAILED DESCRIPTION OF THE PREFERRED EMBODIMENTS

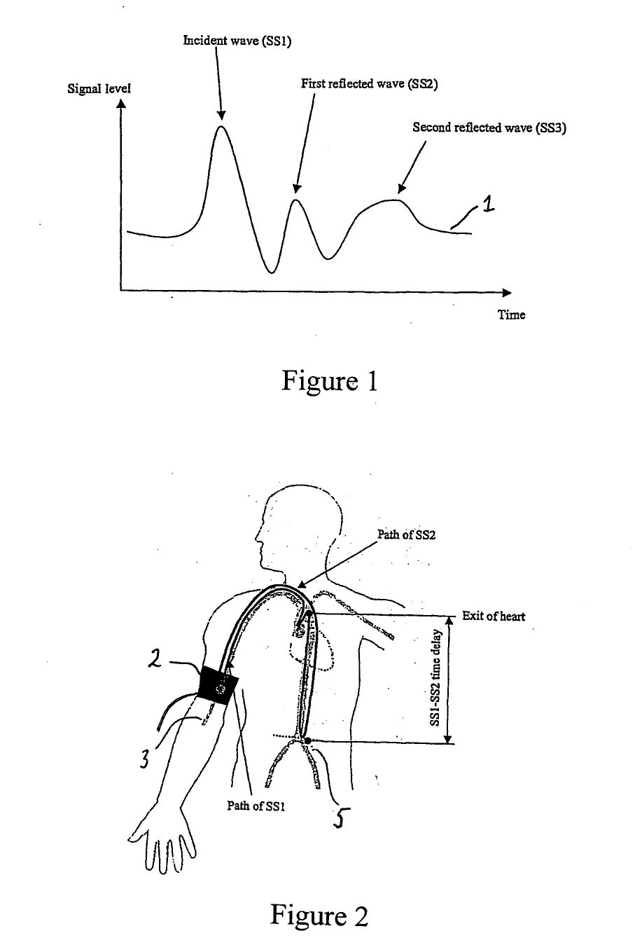

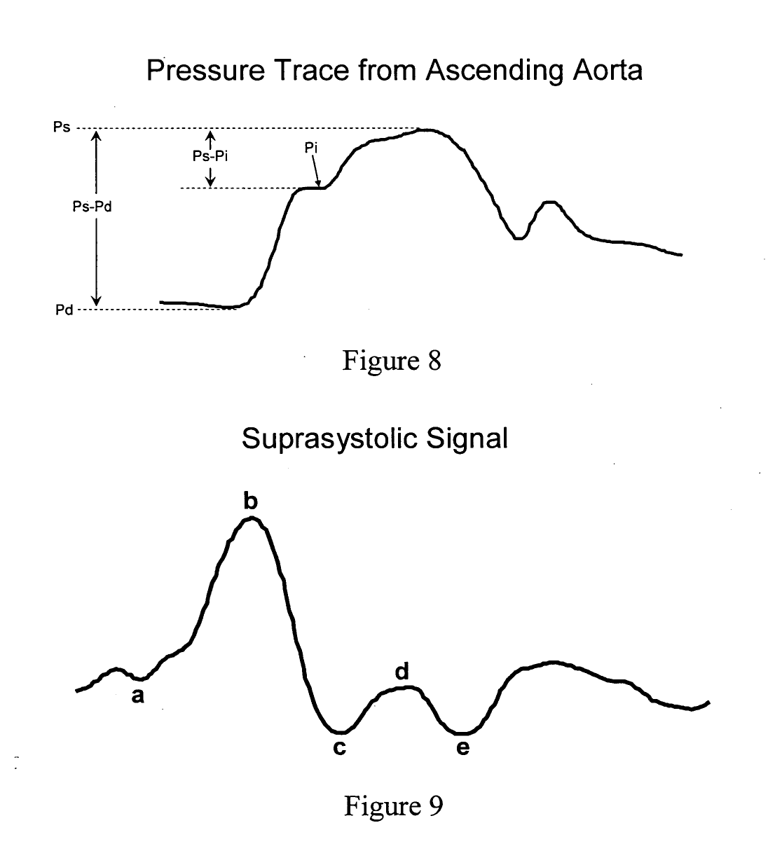

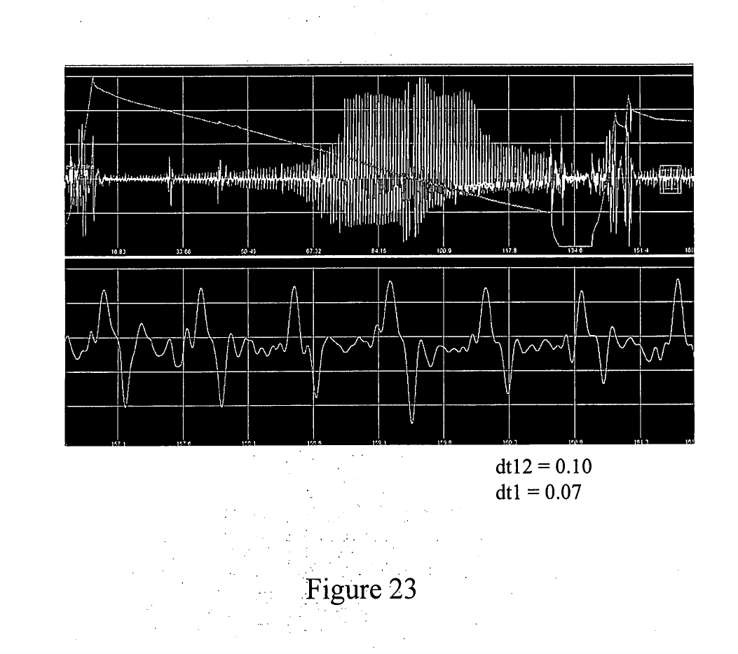

The preferred embodiments of the present invention will now be described with reference to FIGS. 1-27 of the drawings. Identical elements in the various Figures are designated with the same reference numerals.

### BACKGROUND

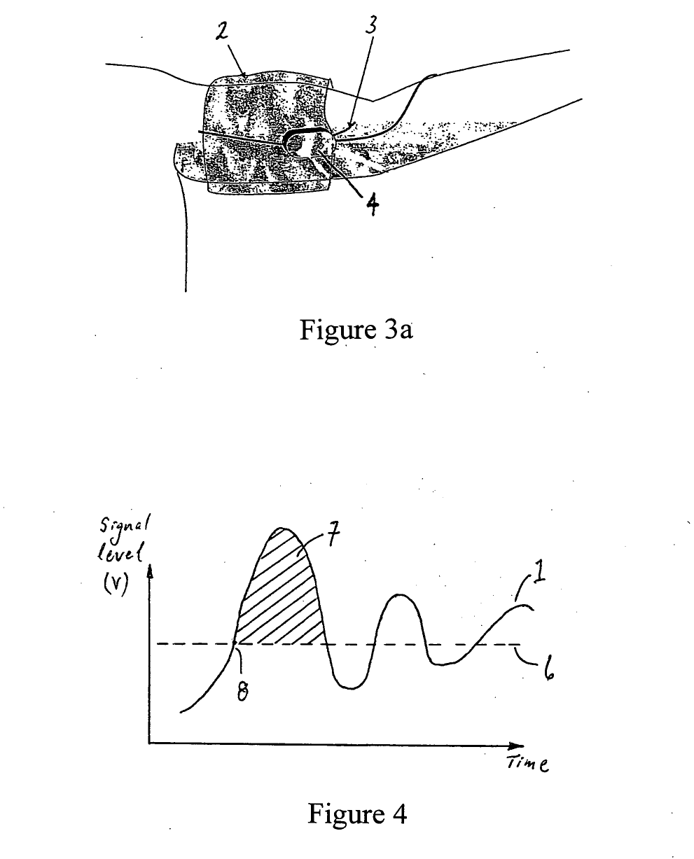

With reference to the drawings and in particular FIG. 1 initially, an idealized supra-systolic signal 1 is shown which has been obtained utilizing the arrangements shown in FIGS. 2 and 3.

The signal shown in FIG. 1 is characteristic of the transduced signal within a patient's brachial artery 3 in the upper aim as a result of applying supra-systolic pressure to the brachial artery utilizing a blood pressure cuff 2 (FIGS. 2 and 3) which has been inflated above the patient's systolic blood pressure (subsequent to a determination being made of the patient's systolic blood pressure). When the blood flow in the brachial artery 3 is occluded, flow related pressure changes are effectively filtered out so that a sensor 4 positioned proximate to the patient's proximal artery may purely measure pressure-induced energy transmissions generated within the cardiovascular system as a result of the heart pumping.

As the heart pumps, pulse pressure waves travel peripherally from the heart through the arteries. These pressure waves reflect back off a surface or other change in impedance. As shown in FIG. 2, the signals sensed at the brachial artery will include the result of a pressure wave traveling directly from the heart (shown as peak or pulse SS1 in FIG. 1) as well as a pressure signal resulting in a reflection of energy traveling from the heart to the distal aorta 5 and back up to the brachial artery (shown as peak or pulse SS2 in FIG. 1). A further peak or pulse or wave (SS3) results from a reflection of the pressure wave off the peripheral circulation and secondary reflections from the distal aorta.

Because the large majority of the energy within the supra-systolic signal of FIG. 1 is outside the frequency range of normal human hearing it is necessary to use a specialized low frequency transducer or sensor 4 (FIG. 3) to obtain the signal of FIG. 1. For example, “wideband” transducers are suitable and in the present application these transducers are often referred to as wideband external pulse (or “WEP”) transducers. WEP transducers may, for example, include piezo-electric sensors capable of converting low frequency mechanical pressure vibrations or fluctuations to an electrical output (voltage) signal. WEP transducer 4 is preferably positioned close to (1.5 to 2 cm) the distal (further from the heart) edge of the blood pressure cuff 2 and aligned with the brachial artery 3 as shown in FIG. 3a.

FIG. 3b illustrates a patient's arm 6 with a blood pressure cuff (Korotkoff cuff) 2 in cross-section. The arm 6 is shown as being surrounded by the partially inflated pressure cuff 2 which comprises an inflatable bladder 8 formed of flexible material. One end 9 of the bladder is wrapped around and secured to itself by means of Velcro® or the like.

A piezoelectric transducer 4 is retained against the surface of the bladder by means of a thin film 10 of synthetic material such as nylon, rayon or the like. The transducer 4 which is retained by the film 10 is positioned such that the transducer receives pressure waves or vibrations from the brachial artery 3.

The previously mentioned WO0205726A and U.S. Pat. No. 5,193,826B both describe methods of determining particular cardiovascular parameters from the output signal of a wideband external pulse transducer. It is known that for example, the magnitude of the SS2 wave is a measure of large arterial tone best assessed by the ratio of the magnitude of the SS1 to SS2 waves. Changes in the SS1:SS2 ratio therefore represent changes in large arterial tone.

Stroke Volume

“Stroke Volume” (SV) is the amount of blood ejected by the heart in a single heartbeat.

As previously mentioned, WO0205726A includes an empirical equation utilizing experimentally determined SS1 and SS2 peak values to calculate stroke volume. “Cardiac Output” is a related cardiovascular parameter indicating the amount of blood pumped by the heart per unit time and is the product of Heart Rate (HR)×Stroke Volume and hence cardiac output may be easily determined once Stroke Volume is known.

It has been discovered and confirmed, according to the invention, that the area beneath the SS1 peak or pulse or portion of the signal as exemplified in FIG. 1 is positively correlated with stroke volume and improvements in cardiac performance. By “positively correlated”, it is meant that stroke volume can be approximated as a function of the area beneath the SS1 pulse. Changes in the area under the SS1 peak or pulse or curve in an individual over time therefore reflect changes in stroke volume and thus the SS1 signal can be used as a monitor of change in stroke volume of an individual or patient over time. As an alternative to area beneath the SS1 peak, it has also been shown that the area beneath a function of the SS1 peak can also provide a good indication of stroke volume. For example, the area beneath a curve which is the square (or other function) of the SS1 peak curve, could be utilized as an indicator of stroke volume.

By utilizing flow probes, it has been determined that the majority of forward flow during a cardiac cycle occurs during the initial stage of systole (the regular contraction of the heart and arteries that drives the blood outward). Analysis of the timing of supra-systolic signals (as shown in FIG. 1) demonstrates that the SS1 signal corresponds to the timing of the peak and forward flow noted with the flow probes. Furthermore, studies have demonstrated that changes in the amplitude of the SS1 signals are consistent with changes in stroke volume.

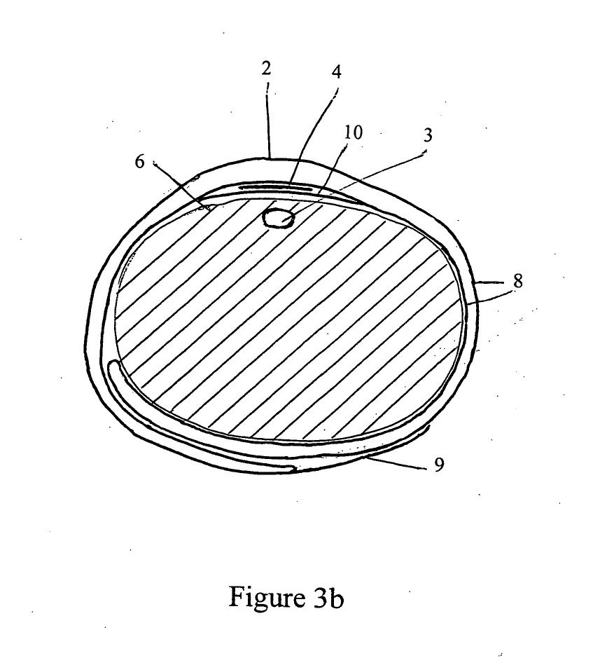

Preferably, the duration of the SS1 curve for the area calculation is the time from the inflection of the SS1 signal (that is, the transition from concave to convex) to the onset of the SS2 signal. FIG. 4 demonstrates the area 7 which must be calculated in which a base line 6 has been inserted at a selected amplitude level through the initial point 8 in the SS1 wave at which it is inflected.

As a result of this discovery of the relationship between the area under the SS1 signal and stroke volume, an empirical equation can be determined or, alternatively, changes in calculated area values in a particular patient over time can be recorded to provide an indication of changes in stroke volume (in comparison to a base value) for that patient. Alternatively, a model of the cardiovascular system may be developed which explains this relationship and serves to predict stroke volume based on SS1 signal data.

Blood Volume

Blood volume is a cardiovascular parameter indicating the amount of blood in a patient's circulatory system. Changes in arterial pressure with breathing (either spontaneous or with a ventilator) are used in clinical practice as a measure of blood volume such that large declines in pressure with ventilation represent volume depletion. Volume depletion leads to less blood returning to the heart and therefore a decline in cardiac output.

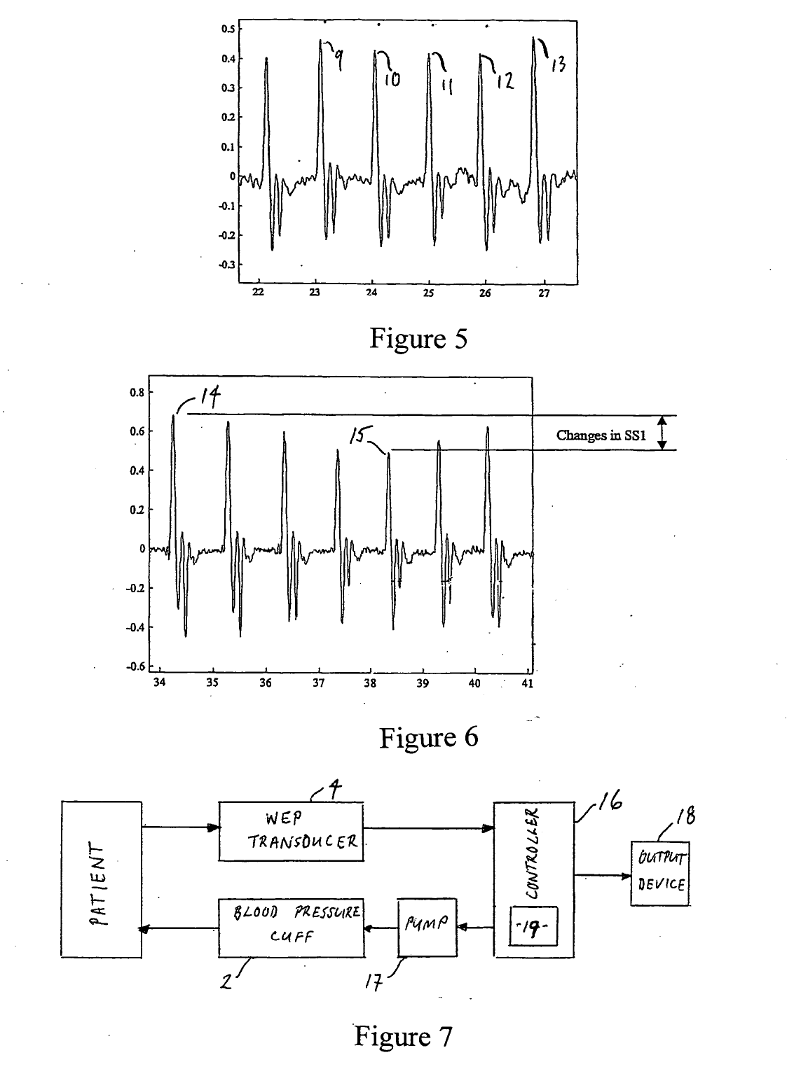

Changes in the magnitude or amplitude of the SS1 signal occur with breathing. It is known that more labored breathing produces a larger decline in the magnitude of the SS1 signal during a breathing cycle (an inhalation followed by an exhalation or vice versa). FIG. 5 shows the effect of normal respiration on the supra-systolic waveform during a typical breathing cycle. It can be seen that SS1 peak 9 is a maximum from start of exhalation and subsequent SS1 peaks 10 and 11 show a gradual reduction in SS1 amplitude whereas peaks 12 and 13 show a gradual increase in SS1 peak amplitude as the patient inhales.

In contrast, FIG. 6 shows a supra-systolic blood pressure signal from a patient whose breathing is labored (for example, the patient may be suffering an asthma attack or be breathing via a ventilator). It can be seen in FIG. 6 that the change in magnitude of the SS1 peak between the maximum peak 14 and minimum amplitude peak 15 is much greater than the example shown in FIG. 5.

It has been discovered that the size of the change in amplitude of the SS1 peak over a respiratory cycle is negatively correlated with blood volume. By “negatively correlate”, it is meant that blood volume can be approximated by a decreasing function of the change in magnitude of the SS1 peak over a breathing cycle. Accordingly, this discovery can be used to empirically determine a relationship or equation which equates the change in amplitude to change in blood volume during the breathing cycle. Alternatively, the measured change in amplitude during a breathing cycle can itself be recorded for comparison with previous or future changes in amplitude for that same patient to generate a trend of changes in blood volume for that patient over time.

Although the examples shown with reference to FIGS. 5 and 6 both utilize supra-systolic blood pressure signals, it should be noted that changes in the magnitude of the SS1 signal with ventilation can also be detected at subsystolic (but greater than diastolic) pressure and even subdiastolic pressure can be used as a measure of change in blood volume.

Both of the above-mentioned discoveries require the obtaining of a signal associated with pressure fluctuations from a peripheral artery of the patient (for example, brachial artery) and the measurement of a feature of that signal. While the obtaining and measurement of the feature of the signal may be carried out manually in the case of measuring the change in amplitude of the SS1 peak, the measurement of these features may be automated. For example, signals from sensor 4 may be amplified, passed through an analog-to-digital converter and input to a computer via data acquisition hardware and analyzed utilizing software such as National Instruments' LabVIEW™ software which provides the ability to not only easily measure the changes in amplitude required for the above blood volume calculation, but also easily enables the selection of a suitable baseline and measurement of the area beneath SS1 to determine stroke volume. It is known that heart rate can also be determined from the SS1 curve and therefore cardiac output may be determined from stroke volume once heart rate has been established. The method for calculating area beneath the SS1 peak may, for example, comprise integrating a determined function between start and end times; components of the SS1 signal—e.g., amplitude and time to achieve peak amplitude—can also be determined.

As shown in FIG. 7, it is possible to automate the process of determining cardiac output or blood volume by utilizing a controller 16 which may comprise hardwired electronic devices or may comprise, for example, a microprocessor running suitable software which receives the output of the WEP transducer 4 and controls inflation/deflation of blood pressure cuff 2 via a controllable air pump 17.

For fast inflation the controller may be programmed (1) to inflate the cuff 2 while monitoring the output of the WEP transducer to determine when the patient's systolic blood pressure is reached, and then (2) to continue to inflate the cuff to between about 25 to 30 mm Hg above the thus determined systolic pressure in order for the controller to obtain the supra-systolic blood pressure signal exemplified in FIG. 1.

The software or hardware within controller 16 (shown as box 19) may then analyze the captured supra-systolic signal to determine such parameters as the peak amplitudes of the various SS1 signals and the area beneath the SS1 signal as well as determining the positioning of the base line for area determination. Software or hardware 19 may then determine the stroke volume and/or blood volume based on the respective measured parameters. For example, software may incorporate an equation correlating the measured parameter to blood volume or stroke volume. Once the appropriate parameter or value has been measured or determined by the software or hardware 19 within or associated with controller 16, the calculated value may be output to an output device such as a display screen or printer 18. Alternatively or in addition, the output device 18 may include storage means for recording the various parameters gleaned from a particular patient's blood pressure signal (and/or the calculated values of stroke volume or blood volume) and software may input the recorded values to determine trends or changes in the parameters or values over time to aid in assessing changes in circulatory physiology.

It is known that an estimate of arterial softness in a patient may be determined based on such cardiovascular parameters as stroke volume and blood volume. Accordingly, the various measurements derived from the suprasystolic waveform (such as area under SS1, the change in peak SS1 value during a breathing cycle, the SS1-SS2 time delay between respective adjacent peaks of SS1 and SS2 and/or ratio of SS1:SS2 peak values) and/or a series of readings taken over time from the same patient may be fed into an appropriately trained neural network which would output a value for arterial softness in the patient under analysis.

Accordingly, at least in its preferred form, the present invention provides a method and apparatus for efficiently and simply measuring cardiac performance in a patient non-invasively.

Arterial Compliance

Arterial compliance refers to the stiffness of arteries. In young healthy people, arteries are compliant so that a volume of blood ejected causes them to distend more for a given pressure. By contrast, stiff arteries (arteries with a low compliance) distend less. Compliance (C) is measured by the change in volume (dV) per unit increase in pressure (dp) (Brinton, Cotter et al. 1997; de Simone, Roman et al. 1999):
C=dV/dp (2) True compliance

Compliance can be measured fairly accurately by stroke volume (SV) divided by pulse pressure (PP) even though the arterial circuit is not a totally closed system (Chemla, Hebert et al. 1998):
C=SV/PP (3) Estimated compliance

Arterial compliance, although important, is not commonly measured in clinical practice, as the measurement, up until now, has been difficult to perform. The aforementioned U.S. patent application Ser. No. 10/221,530, which has been incorporated herein by reference, discloses a technique for obtaining this information non-invasively using a Korotkoff blood pressure cuff.

According to the present invention, it has been found that by measuring aortic waveforms and brachial artery signals concurrently, the relationship therebetween can be understood and a correction function derived. This enables a Brachial Artery Augmentation Index (AAI) and a brachial artery derived AAI to be measured.

The problem with the existing methodologies are that they are technically difficult to use and not easy to readily repeat. The blood pressure cuff/sensor combination is simple to use, provides clear, repeatable data that is easy to analyze, can be cheap to manufacture, and generally will not require trained personnel. It can also be used as a monitor, as it can be left in place wrapped around the patient's arm.

Modeling the Cardiovascular System

The present invention provides a system for measuring peripheral arterial signals, e.g. of the brachial artery, such as the aforementioned occlusive cuff and transducer, for reading pressure fluctuations over the occluded artery, and a processor, receiving the signals from the transducer, and processing these signals to determine distortions present in the waveform transducer waveform with respect to the inferred original aortic waveform.

The method proceeds by occluding a peripheral artery by, for example, inflating a cuff to a supra-systolic pressure, such as 30 mm Hg above a systolic pressure, measuring with an extracorporeal wideband (WEP) transducer a pressure waveform of the peripheral artery, and analyzing the waveform with respect to a model of at least a portion of the cardiovascular system to infer an aortic pressure waveform. This inferred waveform may then be used for a number of purposes, including analyzing cardiac function, analyzing the central and/or peripheral arterial system, or for analyzing the cardiovascular system as a whole.

In order to infer the aortic waveform, it is preferred to model the cardiovascular system to extract features from the waveform having separate meaning or interpretation. These may be orthogonal features or mildly interacting. These features may then be processed with respect to population statistics, in order to normalize the values to obtain an accurate estimate. While it may be possible to avoid the feature extraction, this method potentially results in an improved ability to account for population variability and therefore may provide increased accuracy for a similar number of clinical samples. Likewise, a proper model may allow known pathology of a particular patient to be accounted for, or may allow a proposed diagnosis to be tested with respect to its presumed affect on the cardiovascular system.

Further, extracting a useful low dimensionality parameter (that is, a parameter which has a close correlation to a measurable intrinsic mechanical attribute of the cardiovascular system) from the transducer output, facilitates the use of this parameter as a diagnostic, prognostic, or therapeutic index. Thus, if the parameter corresponds to a therapeutic target of a drug, the parameter may be monitored, and drug use titrated for its desired effect on the cardiovascular system.

It has also been found that various stimuli or stresses can dynamically change the cardiovascular system. For example, a cold stimulus on the hand may produce a peripheral arterial vasoconstriction. Therefore, another optional aspect of the present invention is to measure the response of the cardiovascular system to one or more stimuli or stressors, to produce a characteristic change in the cardiovascular system. The measurements of cardiovascular system are then synchronized with the onset and/or relaxation of the stimulus or stressor. Thus, this provides an additional variable to allow elucidation of parameters of the cardiovascular system (or model thereof), which may be directly useful, and/or useful when analyzed in context. The application of a stressor or stimulus permits distinction between functional parameters (those which vary over time based on extrinsic factors) and fixed parameters (those which are not subject to change over periods of time of interest). Thus, atherosclerosis may be distinguished from stress induced vasoconstriction, even though in a single measurement, these may produce the same waveform, since they may present the same impedance characteristics (e.g., arterial compliance). Once these types of distinctions are made, it is then possible to monitor changes in these responses over time, for example as a result of treatment.

Augmentation Index

Supra-systolic brachial artery signals derived from a wideband sensor placed beneath the distal edge of a blood pressure cuff in apposition with the skin, likewise produce an early and late systolic wave (FIG. 9). The sensor records signals directly from an occluded brachial artery with the blood pressure cuff inflated to 30 mm Hg above systole. See U.S. Pat. No. 5,913,826, WO02/05726, and U.S. Pub. Pat. App. 2003/040675 each of which is expressly incorporated herein by reference.

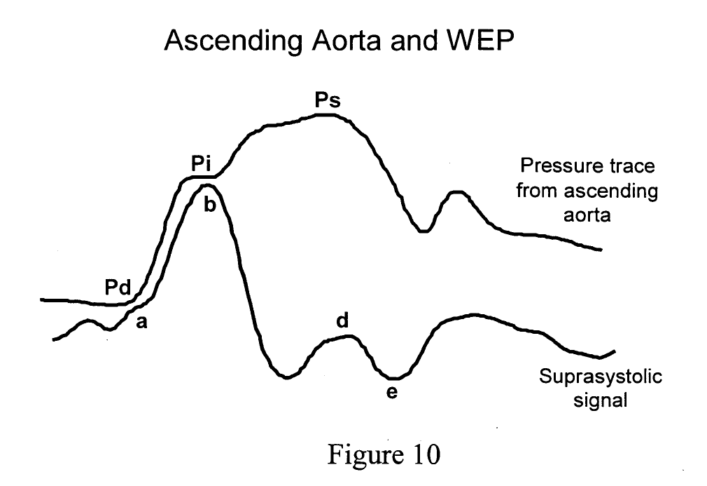

Studies in the cardiac catheterization lab (FIG. 10) demonstrated that the first systolic wave (SS1) corresponds to the first phase of the aortic pressure trace such that the peak of the SS1 (b) corresponds to the Pi of the aortic pressure trace. The late systolic wave SS2 (d) corresponds to the augmentation wave Ps of the aortic pressure trace. From this, it follows that the Augmentation Index can be directly measured by using “de” as the augmentation wave (equivalent to “Ps−Pi”) and using the sum of “ab+de” to be equivalent to “Ps−Pd”.

Thus, the brachial Artery Augmentation Index (AAI) is given by:
AAI=de/(ab+de)×100 (4) Augmentation Index

In a sample of 66 people aged 30-75, Augmentation Index measured in this way provided a value ranging from 5-66%. This range is typical of Augmentation Index measured by other investigators.

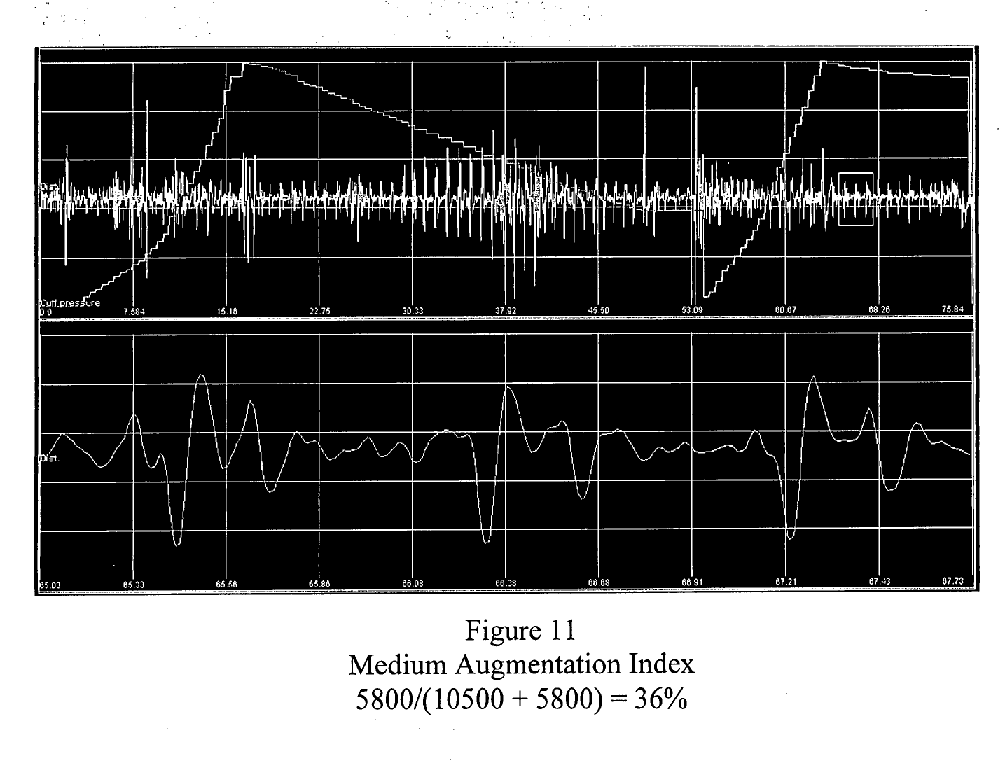

FIGS. 11-15 are screen shots from a computer display showing, in the upper half of the diagram, the pressure wave signal from a wideband external pressure (WEP) transducer and, superimposed thereon, the cuff pressure applied to the Korotkoff arm cuff of a sphygmomanometer along a time axis which is measured in seconds. Thus, in FIG. 11, the time starts at 0.0 seconds and continues to about 76 seconds. As may be seen, the Korotkoff cuff is inflated twice; a first time to determine the approximate systolic pressure and a second time to obtain a supra-systolic signal when the pressure cuff is inflated to a pressure of about 25 to 30 mm Hg above the patient's systolic blood pressure.

The lower part of the diagram shows an expanded view of the WEP transducer signal during the time period indicated by the rectangular box surrounding a portion of the supra-systolic signal along the upper axis. In this case, the box surrounds the portion of the supra-systolic signal which occurs during the 3 second time interval, commencing at approximately the 65 second point along the time scale.

Considering now the formula (4) given above for the brachial artery Augmentation Index, it may be seen that the time distance between the peak of the first reflected wave (SS2) and the following trough (the distance d to e) is approximately 0.58 seconds. Similarly, the distance from the initial trough to the initial peak of the incident wave (SS1) is about 0.105 seconds. Using the formula (4), the augmentation index is calculated to be 36%, which is about average for a healthy, middle aged adult.

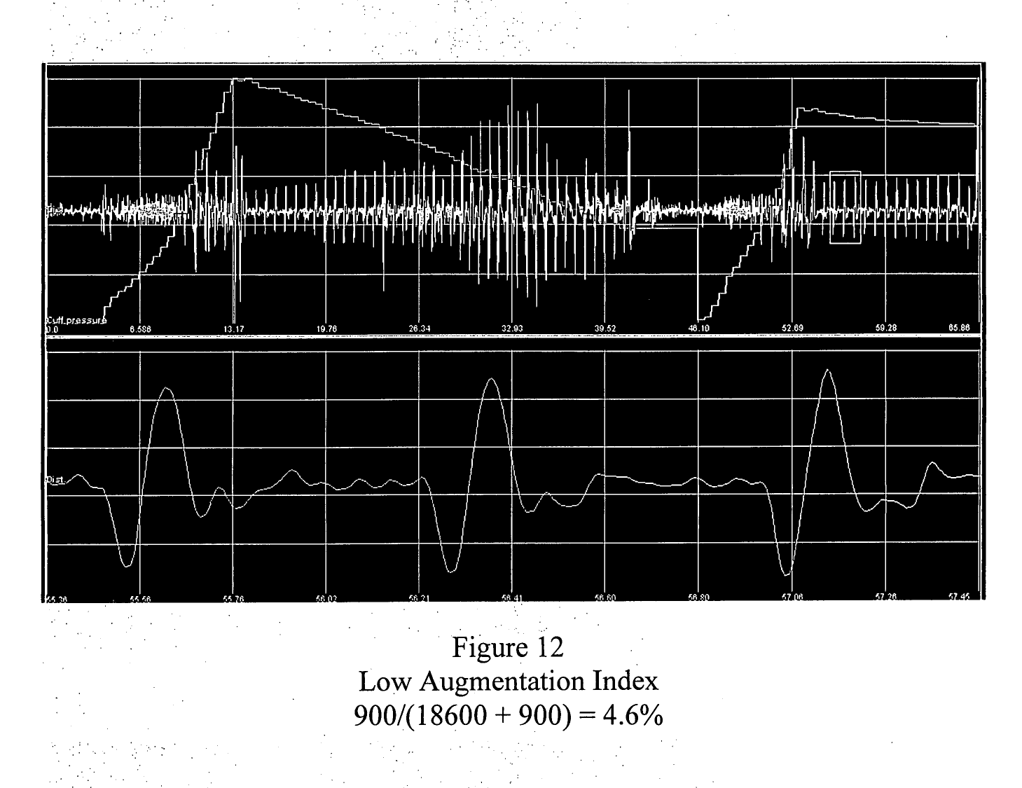

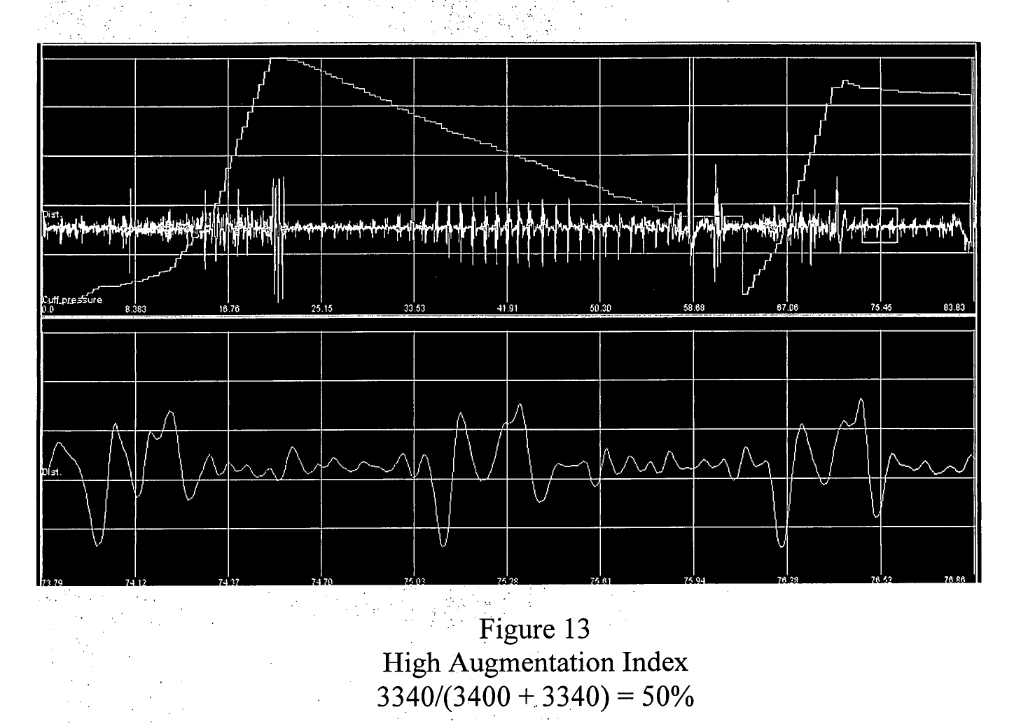

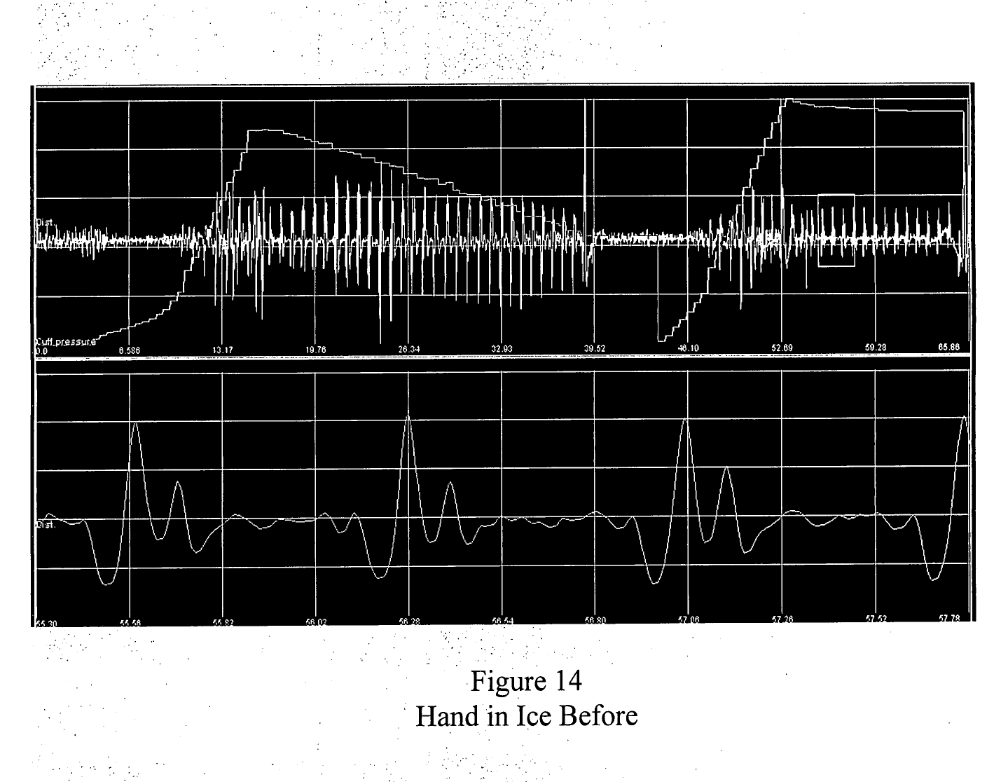

FIGS. 12 and 13 are similar diagrams illustrating a low Augmentation Index of 4.6% and a high Augmentation Index of 50%, respectively.

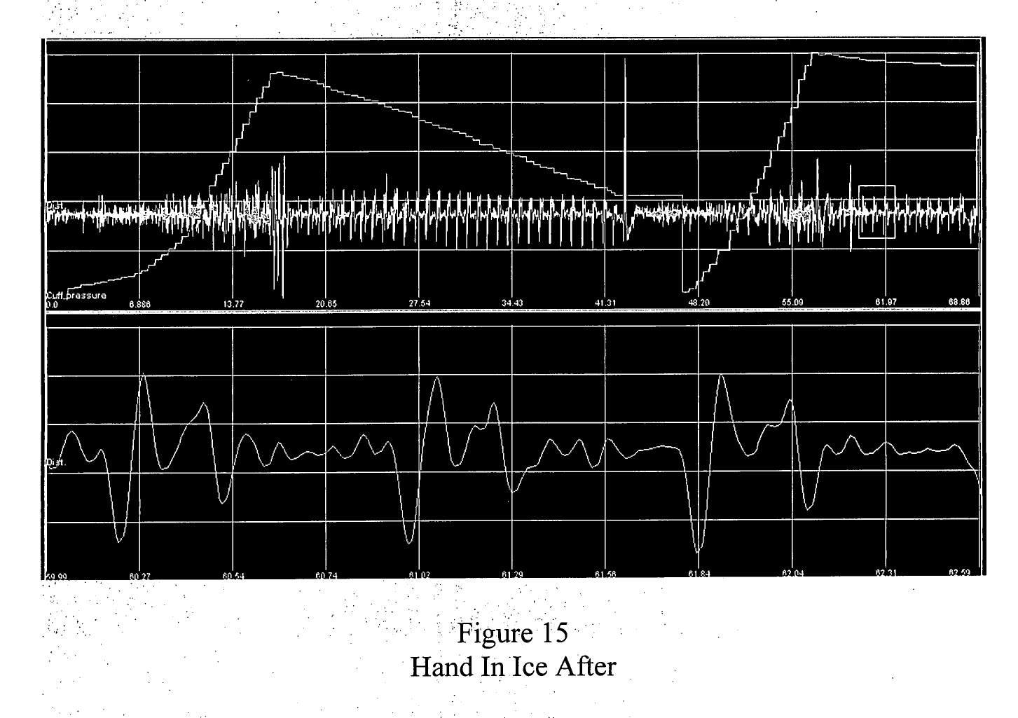

FIGS. 14 and 15 illustrate what happens to the supra-systolic signal when the hand of the arm, to which the Korotkoff cuff has been applied, is placed in ice. In FIG. 14, the supra-systolic signal follows the normal pattern wherein the second reflected wave (SS3) is substantially attenuated from the first reflected wave (SS2). FIG. 15 illustrates that when the hand is placed in ice, causing stress to the adjacent artery, the second reflected wave (SS3) is markedly pronounced. It may be seen, therefore, that the supra-systolic signal reveals useful information relating to a patient's central and peripheral cardiovascular system.

In summary, the present invention provides means for extracting useful parameters of the central and peripheral cardiovascular system performance, without requiring a direct measurement of the pressure waveforms from the heart or aorta.

It is noted that Blank et al., U.S. Pat. No. 5,913,826 refers to use of a modified Windkessel model of circulation, with respect to analysis of the so-called K3 signal. (See also U.S. Pat. No. 5,211,177, expressly incorporated herein by reference). However, these references do not address analysis of external stimuli or stressors, and, for example, Blank et al. suggest that a solution for “white coat hypertension” is to provide a home monitor, and thus to avoid the stress itself, rather than advantageously employ it to perform differential testing.

Cardiac Arrhythmia

When a piezoelectric (WEP) sensor is placed beneath the distal edge of a blood pressure cuff, distinct vascular signals can be detected with the cuff inflated to 30 mm Hg above systolic pressure (supra-systolic signals). These signals have characteristic appearances reflecting the incidence (SS1) and reflective waves (SS2 and SS3). If the cuff is left inflated for 10-12 seconds, a series of pulse signals can be obtained and recorded. This simple non-invasive maneuver provides the equivalence of a rhythm strip used to diagnose arrhythmias on an EKG.

When a typical cardiac arrhythmia occurs, the beat or beats are less effective resulting in an abnormal pulse signal or abnormal interval between beats.

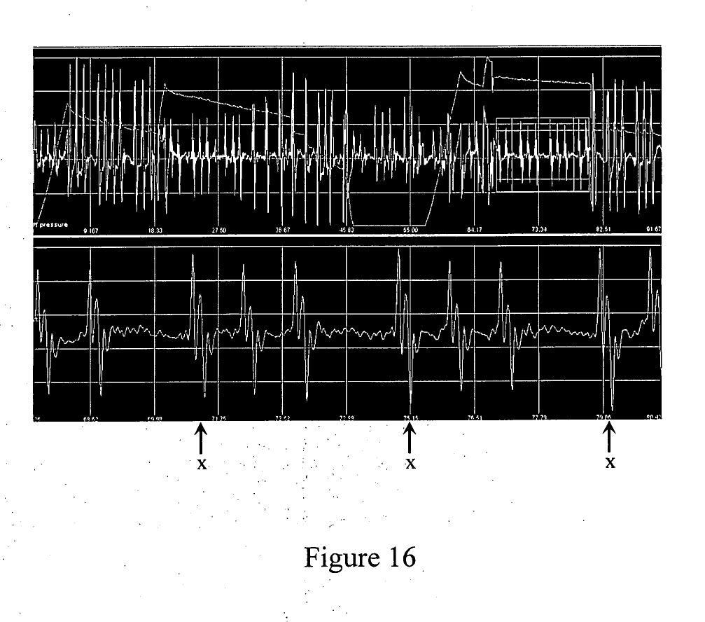

An example of “dropped beats” is shown in FIG. 16. Note the normal characteristic of all beats but the amplitude of the beat following the pause is increased—so called post-ectopic potentiation (FIG. 16 marked “x”).

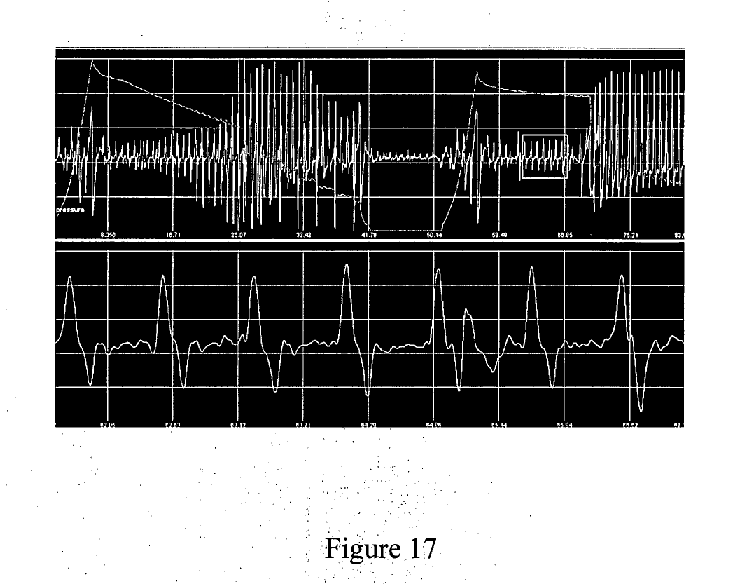

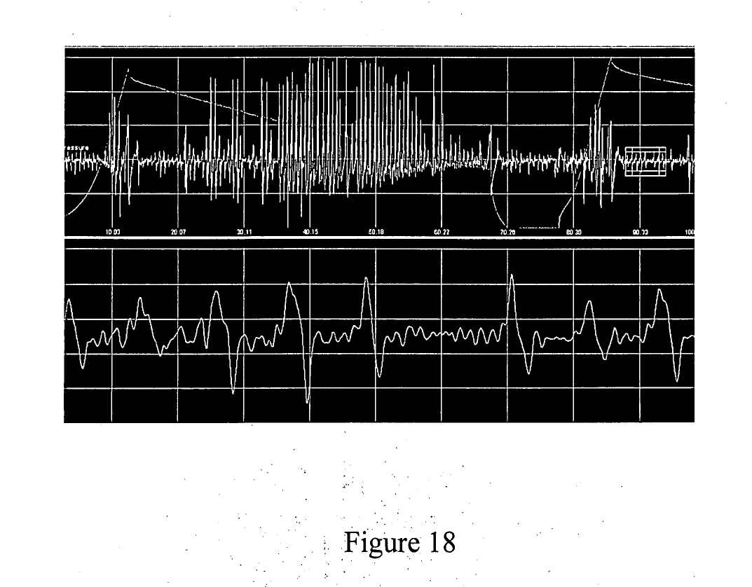

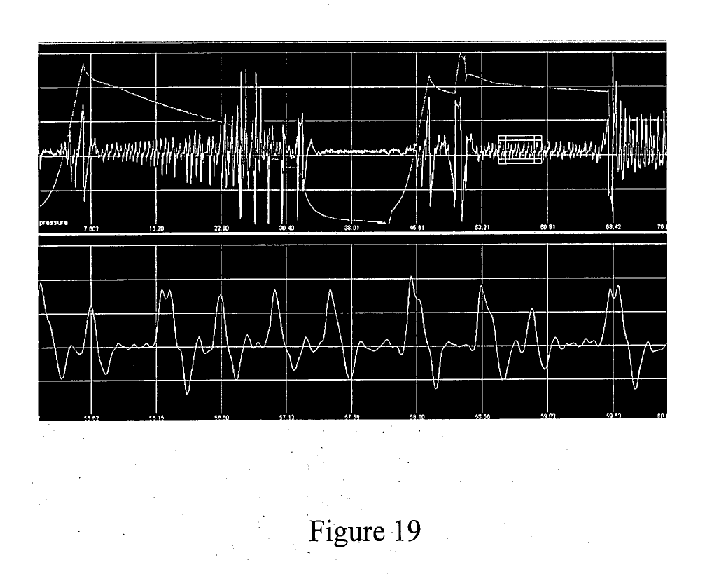

Examples of arterial fibrillation are shown in FIGS. 17-19. Note in FIG. 17 that all beats are similar but beat-to-beat rates vary. In FIGS. 18 and 19, both beat-to-beat rates vary as do the configuration of the waves. This is due to variation in stroke volume/ventricular filling.

Normal beat-to-beat variation occurs and is typical of a healthy heart (so called sinus arrhythmia). Absence of beat-to-beat arrhythmia can be a predictor of heart disease. Beat-to-beat variation in heart rate is measured with software using supra-systolic signals.

The method according to the present invention is not meant to displace the EKG. Rather it is a useful component of the utility of supra-systolic signal analysis as a screening tool for cardiovascular disease in primary care setting. It augments the use of an EKG as this provides a functional analysis of the pulse wave itself.

Heart Failure

Heart failure exists in at least 500,000 people in the United States; these numbers are increasing due to better treatment of ischemic heart disease, aging population, etc. The condition is under diagnosed, under treated and places a huge burden on the health care industry.

Diagnosis and management of treatment often entails expensive cardiac technology. The most frequently used is echocardiography. These machines cost $200,000 each, require expert technician to use and physicians to interpret the studies. More expensive or invasive tests are also used. There is thus a need for a simple technology to assess heart failure in the primary care environment or for routine management by cardiologists. The use of supra-systolic signal analysis can provide cheap simple non-invasive assessment of cardio-vascular function including insight into the existence of heart failure or the propensity to develop heart failure.

There are several forms of heart failure:
1. Systolic heart failure wherein the left ventricle loses contractile strength. The heart doesn't pump well and cardiac output falls.
2. The other common category is diastolic heart failure wherein the heart becomes stiff. It doesn't relax well and is subject to fluid overload, pulmonary edema and acute heart failure.

Evidence of both forms of heart failure can be detected or assessed with supra-systolic wave analysis.

When the heart ejects blood into the aorta, a pulse wave enters the large vessels and is reflected back off the distal aorta. The reflectance wave becomes more prominent and returns more rapidly with aging or degenerative diseases of the large arteries. This results in a resistance to forward flow of blood.

As has been described above, the amplitude of the forward and reflective waves and the duration between them can be accurately determined by analyzing signals obtained from a sensor placed over the skin adjacent to the brachial artery. The sensor is positioned beneath the distal edge of a blood pressure cuff wrapped around the arm. With the blood pressure cuff inflated 30 mm Hg above systolic pressure for 10-12 seconds, a series of pulse recordings are obtained. The average of these beats provides a mean value for the SS1 and SS2 waves. The characteristics of these waves can be used to diagnose systolic heart failure and the propensity to develop heart failure.

Systolic Heart Failure

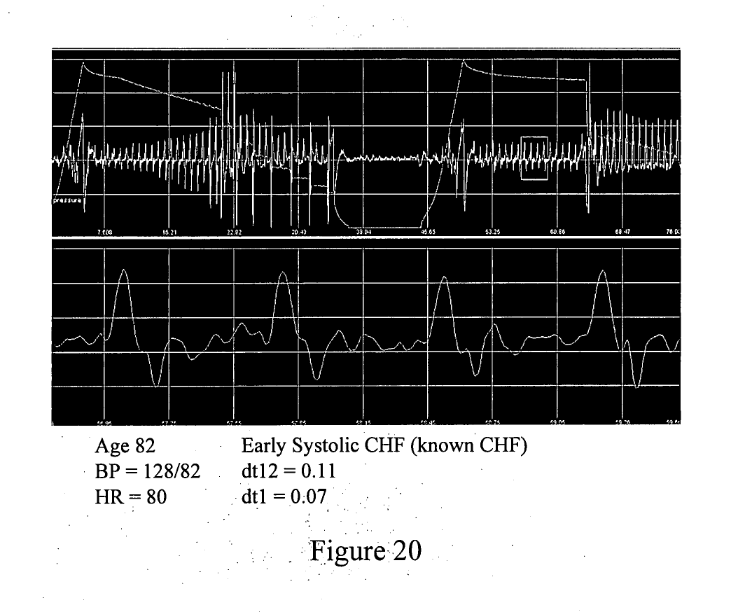

Typical tracings shown in FIGS. 20-23 illustrate supra-systolic signals from patients with systolic heart failure.

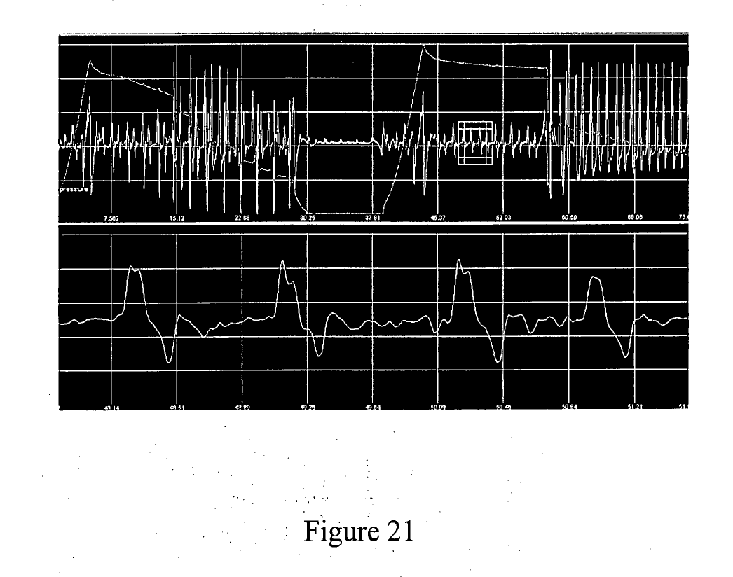

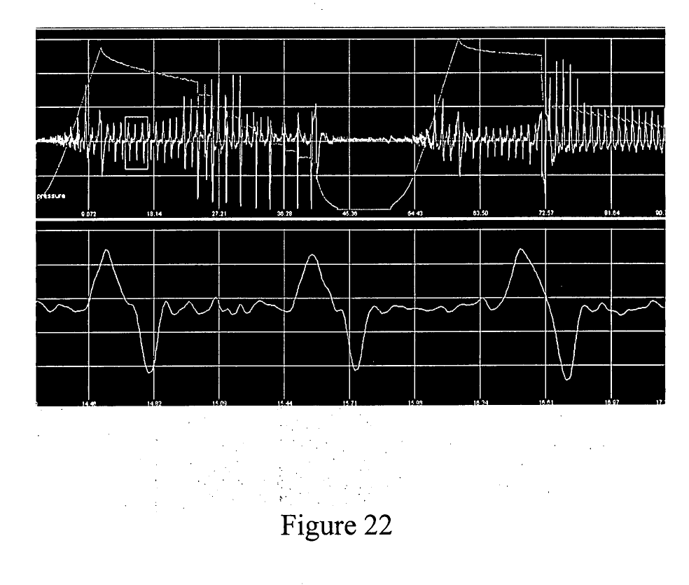

The pumping strength of the heart decreases thus producing a less intense SS1 and the reflection wave (SS2) is either absent or incorporated into the descending portion of the SS1 (FIGS. 21-23). Typically, this SS2 is incorporated into the down slope approximately halfway down the slope with a dt1-2 of 0.8-0.11 second. (dt12 is the delay between the peak of SS1 and SS2). The duration of the upstroke of SS1 (dt1) may be prolonged and the amplitude of the SS1 wave decreased. The SS1 wave may be biphasic (FIG. 21).

Diastolic Heart Failure

As large arteries harden, the pulse wave velocity increases and the amplitude of the reflection wave increases. This results in a shortening of the SS1-SS2 period—the period during which the majority of blood is ejected from the ventricle. As the period for ventricular emptying shortens, this places additional strain on the left heart resulting in left ventricular hypertrophy. Eventually, the duration of ventricular emptying gets so limited that the heart fails. Thus, the duration dt1-2 can be used as a predictor of the likelihood of developing heart failure or secondly, as a marker that the patient has heart failure. When the dt1-2 is 0.10 seconds or less, it is likely that the heart will fail or heart failure is already established. In young patients, dt12 may be 0.15-0.2 seconds.

Two factors in ventriculo-vascular coupling which adversely affect ventricular emptying are the duration of the waves and the amplitude of the SS2. The greater the amplitude of the SS2, the greater the impediment to forward flow. The shorter the dt12, the less time there is for ventricular emptying. Thus, a short dt12 and high amplitude SS2 foretell adverse ventricular emptying, ventricular strain and impending diastolic heart failure.

Importance of dt1-2

The duration or time lapse between the peaks of the two supra-systolic peaks SS1 and SS2 is an important measurement for two reasons: first, as a measure of pulse wave velocity, and second, as a measure of the adverse effect of the reflection wave on ventriculo-vascular coupling and ventricular emptying.

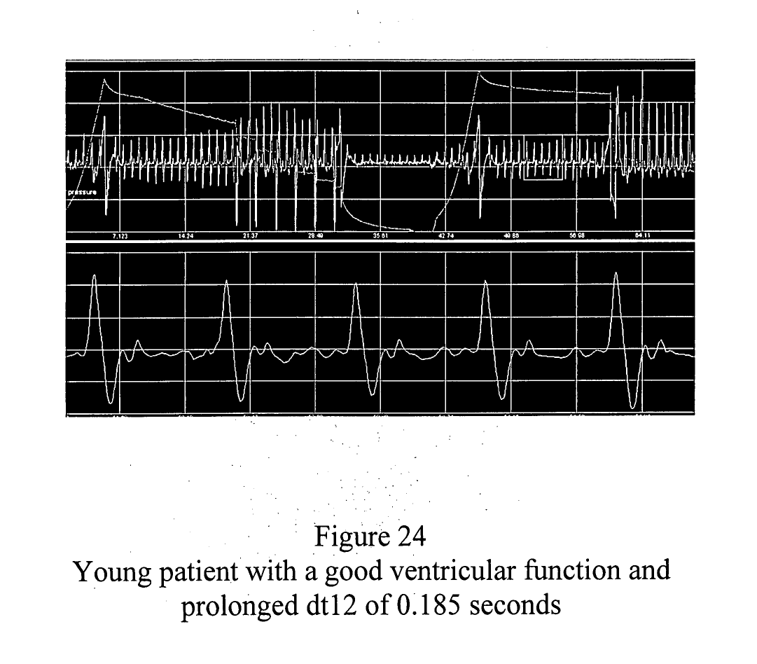

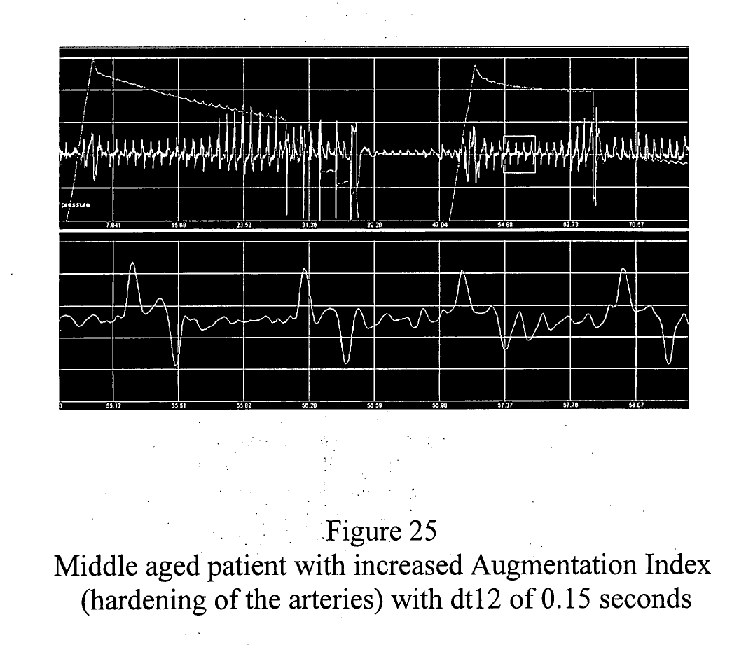

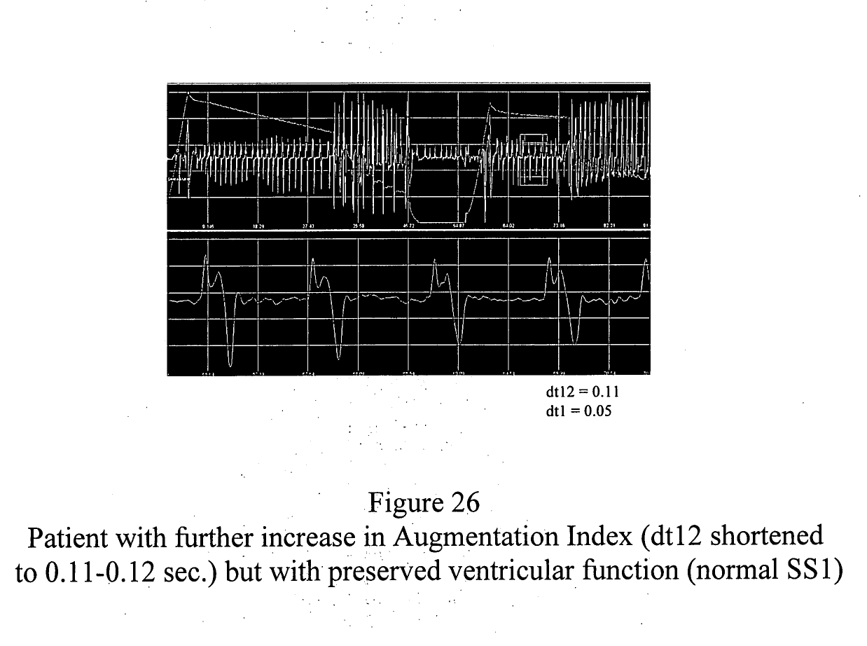

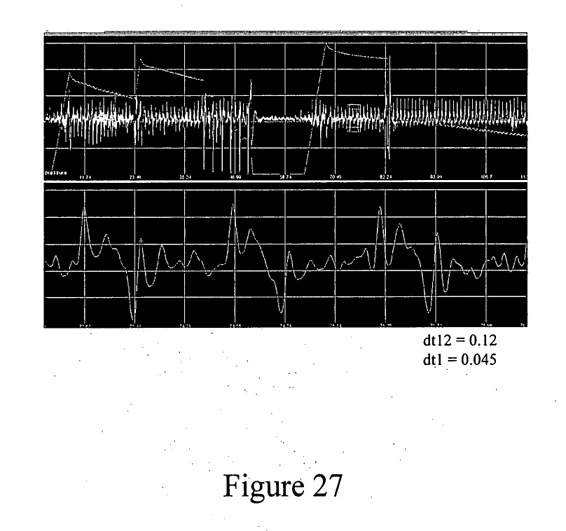

Four tracings are shown for comparison (FIGS. 24-27). FIG. 24 shows the expanded WEP tracing for a young patient having good ventricular function. In this case, the period dt1-2, between the peak of the incident wave SS1 and the first reflected wave SS2 is a prolonged 0.185 seconds. In contrast, a middle-aged patient with increased Augmentation Index (hardening of the arteries) may have a dt1-2 of about 0.15 seconds (FIG. 25). An increase in the Augmentation Index and a shortening of dt1-2, but with preserved ventricular function (i.e., a normal SS1 peak in the supra-systolic wave) is illustrated in FIG. 26 (dt1-2=0.11 sec.) and in FIG. 27 (dt1-2=0.12 sec.).

Changes in supra-systolic signals with exercise can be used in two ways. First, the patient's response to acute exercise can be assessed. Normal individuals exhibit an increase in the amplitude of the SS1 signal consistent with an increase in stroke volume, a decrease in the time to generate the SS1 (dt1) consistent with increased cardiac contractility and a decrease in their Augmentation Index (AI) representing arterial dilatation. Second, physical training with conditioning results in an improvement in arterial compliance which manifests as a reduction in AI. These changes can be used to assess (1) cardiovascular fitness and (2) the cardiovascular benefits of an exercise prescription.

The preceding preferred embodiments are illustrative of the practice of the invention. It is to be understood, however, that other expedients known to those skilled in the art, or disclosed herein, may be employed without departing from the spirit of the invention or the scope of the claims.

What is claimed is:

1. A method for determining a cardiovascular status of a mammal having a cardiovascular system that includes a peripheral artery, comprising the steps of:
(a) measuring a sequence of cardiac pulse waveforms associated with the peripheral artery and representing a plurality of cardiac ejection cycles;
(b) analyzing the waveforms with respect to at least one parameter of the cardiovascular system, said parameter being selected from the group consisting of Augmentation index (AI), cardiac performance, and cardiac stroke volume; and
(c) generating an output based on said analysis.

2. The method defined in claim 1, wherein the peripheral artery is a brachial artery and wherein the cardiac pulse waveforms are restricted to the initial peak waveform (SS1) following each cardiac ejection cycle.

3. The method defined in claim 2, wherein the brachial artery is occluded by a blood pressure cuff inflated to a supra-systolic pressure.

4. The method defined in claim 3, wherein the blood pressure cuff is inflated to a pressure in the range of substantially 25 to 30 mm Hg above systole.

5. The method defined in claim 1, further comprising the step of applying a stress to at least a portion of the cardiovascular system of the mammal as said waveforms are measured.

6. The method defined in claim 5, wherein the peripheral artery is a brachial artery and wherein the step of applying a stress includes applying cold to at least a portion of a limb which includes the brachial artery.

7. The method defined in claim 2, wherein the parameter is the Aortic Augmentation Index (AAI).

8. The method defined in claim 7, wherein the Aortic Augmentation Index (AAI) is determined from the pressure waveform having an incident wave (SS1) and a first reflected wave (SS1), by dividing the descent time of the first reflected wave (SS2) by the rise time of the incident wave (SS1) plus the descent time of the first reflected wave (SS2).

9. The method defined in claim 2, wherein the parameter is stroke volume of the heart.

10. The method defined in claim 9, wherein the stroke volume is determined from the pressure waveform having an incident wave (SS1) with a peak by calculating the area under the peak.

11. An apparatus for determining a cardiovascular status of a mammal having a cardiovascular system that includes a peripheral artery, said apparatus comprising:
(a) a low frequency, wideband pressure transducer for measuring over time a sequence of cardiac pressure waveforms associated with a peripheral artery of a patient; and
(b) a processor, receiving an output of said transducer, and analyzing the cardiac pressure waveform to with respect to a change in a parameter of a model representing the cardiovascular system of the mammal and generating an output bases on the analysis.

12. The apparatus defined in claim 11, wherein the peripheral artery is a brachial artery and said apparatus further comprises a blood pressure cuff adapted to surround an arm of a patient with the brachial artery and to press the transducer against the brachial artery.

13. The apparatus defined in claim 12, wherein the apparatus further comprises an air pump and a controller for the air pump for inflating the cuff to supra-systolic pressure, wherein the cardiac waveforms are produced when the cuff is at supra-systolic pressure.

14. The apparatus defined in claim 11, wherein the model of the cardiovascular system has at least one intrinsic mechanical attribute and the processor is programmed to determine a parameter of the pressure waveform based on this attribute.

15. A blood pressure cuff comprising:
(a) an elongate bladder suitable for wrapping around the arm of a patient;
(b) an air pump adapted to be connected to the bladder for inflating the bladder to a desired pressure; and
(c) a low frequency wideband external pulse transducer attached to the bladder in a position such that it is situated adjacent to the brachial artery when the bladder is in place on the arm of a patient.

16. The blood pressure cuff defined in claim 15, wherein the transducer is positioned substantially in the range of 1.0 and 1.5 cm from the distal border of the bladder when it is in place on the arm of a patient.

17. The blood pressure cuff defined in claim 15, wherein the transducer is a piezo-electric transducer.

18. The blood pressure cuff defined in claim 15, wherein the bladder is made of flexible material and wherein the transducer is disposed outside the flexible material.

19. The blood pressure cuff defined in claim 18, wherein the transducer is attached to the outside surface of the flexible material.

20. The blood pressure cuff defined in claim 15, wherein the transducer is arranged between the bladder and a thin film of protective material.

21. A method of non-invasively obtaining information about heart stroke volume of a patient, said method comprising the steps of:
i) obtaining a signal indicative of supra-systolic blood pressure amplitude with time from a peripheral artery of the patient with the peripheral artery's blood flow occluded;
ii) measuring the area beneath a first major peak in the signal, or a function of this signal, and above a base line; and
iii) determining, based on the measured area, at least one of the stroke volume of the patient and a change in stroke volume over time.

22. The method defined in claim 21, wherein the step of obtaining a signal includes positioning a wideband external pulse transducer proximate to said peripheral artery.

23. The method defined in claim 22, wherein the step of obtaining a signal includes applying pressure to said peripheral artery.

24. The method defined in claim 23, wherein the peripheral artery is the brachial artery and pressure is applied by a blood pressure cuff placed around the patient's arm.

25. The method defined in claim 24, wherein said wideband external pulse transducer is positioned beneath the distal edge of the blood pressure cuff.

26. The method defined in claim 24, wherein the blood pressure cuff is inflated to a pressure of about 25 to 30 mm Hg above the patient's systolic blood pressure.

27. The method defined in claim 21, wherein the base line is selected at an amplitude which passes through an initial inflection point in the first major peak.

28. The method defined in claim 21, wherein the method is repeated a plurality of times to obtain a plurality of area values which are compared to determine a change in stroke volume value for the patient.

29. A method of non-invasively determining a change in blood volume in a patient comprising the steps of:
i) obtaining a signal indicative of blood pressure from a peripheral artery of the patient over at least one inspiratory/expiratory breathing cycle of the patient, the signal containing a repeating sequence of groups of pulses including a first major peak in each group;
ii) measuring a change in the amplitude of the first major peak between different groups of pulses; and
iii) determining, based on the measured change in amplitude, the change in blood volume of the patient.

30. The method defined in claim 29, wherein the step of obtaining a signal indicative of blood pressure comprises obtaining a signal indicative of supra-systolic blood pressure from the patient's peripheral artery by occluding the peripheral artery's blood flow.

31. The method defined in claim 29, wherein the step of obtaining a signal indicative of blood pressure comprises obtaining a signal indicative of subs-ystolic blood pressure from the patient's peripheral artery by applying a pressure to the patient's peripheral artery which is below the patient's systolic blood pressure but above the patient's diastolic blood pressure.

32. The method defined in claim 29, wherein the step of obtaining a signal indicative of blood pressure comprises obtaining a signal indicative of sub-diastolic blood pressure from the patient's peripheral artery by applying a pressure to the patient's peripheral artery which is below the patient's diastolic blood pressure.

33. The method defined in claim 29, wherein the step of measuring a change in amplitude comprises determining the difference between the maximum amplitude and minimum amplitude of the first major peaks from all the groups of pluses within the signal in at least one complete inspiratory/expiratory breathing cycle of the patient.

34. The method defined in claim 29, wherein the step of obtaining a signal includes positioning a wideband external pulse transducer proximate to said peripheral artery.

35. The method defined in claim 29, wherein the step of obtaining a signal includes applying pressure to said peripheral artery.

36. The method defined in claim 35, wherein the peripheral artery is the brachial artery and pressure is applied by a blood pressure cuff placed around the patient's arm.

37. The method defined in claim 36, wherein said wideband external pulse transducer is positioned beneath the distal edge of the blood pressure cuff.

38. The method defined in claim 36, wherein the blood pressure cuff is inflated to a pressure of about 25 to 30 mm Hg above the patient's systolic blood pressure.

39. Apparatus for non-invasively determining stroke volume of a patient comprising:
means for obtaining a signal indicative of supra-systolic blood pressure amplitude with time from a peripheral artery of the patient with the peripheral artery's blood flow occluded;
measuring means for measuring the area beneath a first major peak in the signal or a function of the signal and above a base line; and
determining means which, based upon the measured area, determines the stroke volume of the patient.

40. The method defined in claim 39, wherein the means for obtaining a signal include a wideband external pulse transducer positioned proximate to said peripheral artery.

41. The method defined in claim 40, wherein the means for obtaining a signal include means for applying pressure to said artery.

42. The method defined in claim 41, wherein the peripheral artery is the brachial artery and the means for applying pressure comprises a blood pressure cuff placed around the patient's arm.

43. The method defined in claim 42, wherein the wideband external pulse transducer is positioned beneath the distal edge of the blood pressure cuff.

44. The method defined in claim 42, wherein the blood pressure cuff is inflated to a pressure of about 25 to 30 mm Hg above the patient's systolic blood pressure.

45. The method defined in claim 39, further comprising level selection means for selecting the level of the base line at an amplitude which passes through an initial inflection point in the first major peak.

46. The method defined in claim 42, further comprising control means for automatically inflating the blood pressure cuff, receiving the signal output by the wideband external pulse transducer, selecting the level of the base line in the signal, measuring the area beneath the first major peak in the signal and above the line, determining the cardiac output based on the measured area, and outputting the determined cardiac output value.

47. The method defined in claim 46, further comprising means to record separate measurements of area beneath the first major peak for a particular patient on a plurality of occasions so that a change in cardiac output value for the patient may be determined for the patient by comparing the recorded values.

48. Apparatus for non-invasively determining a change in blood volume in a patient comprising:
means for obtaining a signal indicative of blood pressure from a peripheral artery of the patient over at least one inspiratory/expiratory breathing cycle of the patient, the signal containing a repeating sequence of groups of pulses including a first major peak in each group;
measuring means for measuring a change in the amplitude of the first major peak between different groups; and
determining means which determines the change in blood volume of the patient based on the measured change in amplitude of the first major peak.

49. The method defined in claim 48, wherein the measuring means determines the difference between the maximum amplitude and minimum amplitude of the first major peaks from all of the pulse groups within the signal in at least one inspiratory/expiratory breathing cycle of the patient.

50. The method defined in claim 48, wherein the means for obtaining a signal indicative of blood pressure includes means for applying pressure to the patient's peripheral artery.

51. The method defined in claim 50, wherein the means for obtaining a signal includes a wideband external pulse transducer which is positioned proximate to said peripheral artery.

52. the method defined in claim 51, wherein the peripheral artery is the brachial artery and pressure is applied by a blood pressure cuff placed around the patient's arm.

53. The method defined in claim 52, wherein the wideband external pulse transducer is positioned beneath the distal edge of the blood pressure cuff.

54. The method defined in claim 52, wherein the blood pressure cuff is inflated to a pressure of about 25 to 30 mm Hg above the patient's systolic blood pressure.

55. The method defined in claim 51, further comprising control means for automatically inflating the blood pressure cuff to a desired pressure, receiving the signal output by the wideband external pulse transducer, measuring the change in amplitude of the first major peak between different pulse groups, determining change in blood volume of the patient based on the measured change in amplitude, and outputting the determined change in blood volume value.

56. A method for diagnosing heart disease of a patient having a cardiovascular system that includes a peripheral artery, said method comprising the steps of:
(a) measuring a signal indicative of supra-systolic blood pressure amplitude with time from the peripheral artery of the patient with the peripheral artery's blood flow occluded, said signal thereby indicating the presence and amplitude of heartbeats;
(b) automatically determining, from the signal, any variation in at least one of beat-to-beat rate and beat-to-beat amplitude; and
(c) producing a diagnosis of heart disease based on said variations, if any.

57. The method defined in claim 56, wherein the signal is measured continuously for at least 10 seconds.

58. The method defined in claim 56, wherein the absence of beat-to-beat variations is indicative of heart disease.

59. The method defined in claim 56, wherein the blood pressure cuff is inflated to a pressure in the range of substantially 25 to 30 mm Hg above systole.

60. A method for diagnosing the propensity of heart failure in a patient having a cardiovascular system that includes a peripheral artery, said method comprising the steps of:
(a) measuring a signal indicative of supra-systolic blood pressure amplitude with time from the peripheral artery of the patient with the peripheral artery's blood flow occluded, said signal thereby indicating each forward (SS1) and reflective (SS2) wave resulting from each heartbeat;
(b) determining at least one of the amplitude of the SS2 wave and the delay time dt1-2 between the peak of the SS1 wave and an immediately following SS2 wave; and
(c) producing a diagnosis of heart disease based on the information determined in step (b).

61. The method defined in claim 60, wherein the signal is measured continuously for at least 10 seconds.

62. The method defined in claim 60, wherein the presence of an excessive amplitude of the SS2 wave is indicative of heart disease.

63. The method defined in claim 60, wherein a delay time dt1-2 substantially equal to or less than 0.1 second is indicative of heart disease.

64. The method defined in claim 60, wherein the blood pressure cuff is inflated to a pressure in the range of substantially 25 to 30 mm Hg above systole.
